(19) United States
(12) Patent Application Publication
(10) Pub. No.: US 2006/0235232 A1
(43) Pub. Date: Oct. 19, 2006

(54) Catalysts for gas phase oxidations

(75) Inventors: Samuel Neto, Dresden (DE); Jurgen Zuhlke, Speyer (DE); Sebastian Storck, Mannheim (DE); Frank Rosowski, Mannheim (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 10/565,731

(22) PCT Filed: Jul. 30, 2004
(86) PCT No.: PCT/EP04/08596

§ 371 Date: Jan. 24, 2006

(30) Foreign Application Priority Data
Aug. 1, 2003 (DE) 103 35 346.1

(51) Int. Cl. B01J 27/19; B01J 27/188; B01J 27/198; B01J 27/00; B01J 31/00; C07D 307/89; C07C 51/255

(52) U.S. Cl. 549/248; 502/208; 502/209; 502/210; 502/211; 502/159; 562/410

(57) ABSTRACT

A catalyst for gas-phase oxidations which comprises an inert support and a catalytically active composition comprising transition metal oxides applied thereto, or a precatalyst, is described. The (pre)catalyst is obtained by treating the inert support with an aqueous suspension or solution of the transition metal oxides or their precursor compounds, where the suspension contains a binder dispersion and the binder is a copolymer of an α-olefin and a vinyl-C₂-C₄-carboxylate whose vinyl C₂-C₄-carboxylate content is at least 62 mol %.

The present invention relates to a catalyst for gas-phase oxidations, which comprises an inert support and a catalytically active composition comprising transition metal oxides which has been applied thereto with the aid of a polymeric binder, and also to a process for the catalytic gas-phase oxidation of aromatic hydrocarbons to carboxylic acids and/or carboxylic anhydrides using the catalyst.

Many carboxylic acids and/or carboxylic anhydrides are prepared industrially by catalytic gas-phase oxidation of aromatic hydrocarbons such as benzene, the xylenes, naphthalene, toluene or durene in fixed-bed reactors, preferably shell- and-tube reactors. In this way, it is possible to obtain, for example, benzoic acid, maleic anhydride, phthalic anhydride, isophthalic acid, terephthalic acid or pyromellitic anhydride. In general, a mixture of a gas comprising molecular oxygen, for example air, and the starting material to be oxidized is passed through a large number of tubes which are arranged in a reactor and in which a bed of at least one catalyst is present. To regulate the temperature, the tubes are surrounded by a heat transfer medium, for example a salt melt.

Catalysts which have been found useful for these oxidation reactions are coated catalysts in which the catalytically active composition is applied in the form of a shell to an inert support material such as steatite. The catalytically active constituents of the catalytically active composition of these coated catalysts are generally titanium dioxide (in the form of its anatase modification) together with vanadium pentoxide. Furthermore, small amounts of many other oxidic compounds which act as promoters to influence the activity and selectivity of the catalyst can be present in the catalytically active composition.

To produce such coated catalysts, a solution or suspension of the constituents of the active composition and/or their precursor compounds in an aqueous and/or organic solvent is sprayed onto the support material at elevated temperature until the desired proportion of active composition, based on the total weight of the catalyst, has been reached.

To improve the quality of the coating, it has become standard practice in industry to add organic binders, preferably copolymers, advantageously in the form of an aqueous dispersion, of vinyl acetate-vinyl laurate, vinyl acetate-acrylate, styrene-acrylate or vinyl acetate-ethylene, to the suspension. In addition, the addition of binder has the advantage that the active composition adheres well to the support, so that transport and installation of the catalyst are made easier.

In the thermal treatment at from ≧200 to 500° C., the binder is removed from the applied layer by thermal decomposition and/or combustion. The thermal treatment is usually carried out in situ in the oxidation reactor.

EP-A 0 744 214 discloses a supported catalyst which is obtained by applying a surface coating onto an inert support body. Organic binders mentioned are vinyl acetate-vinyl laurate, vinyl acetate/acrylate, styrene-acrylate, vinyl acetate-maleate and vinyl acetate-ethylene.

DE-A 197 17 344 describes a process for producing catalysts in which a mixture of oxides is milled in the presence of water and subsequently applied to support bodies. Organic binders mentioned are vinyl acetate-vinyl laurate, vinyl acetate-acrylate, styrene-acrylate, vinyl acetate-maleate and vinyl acetate-ethylene.

U.S. Pat. No. 4,397,768 describes a catalyst for preparing phthalic anhydride. The active composition is applied to an inert support with the aid of organic binders such as vinyl acetate-vinyl laurate, vinyl acetate-acrylate, styrene-acrylate, vinyl acetate-maleate or vinyl acetate-ethylene.

DE-A 198 24 532 discloses a binder for producing coated catalysts which comprises a polymer of ethylenically unsaturated acid anhydrides and an alkanolamine having at least 2 OH groups, not more than 2 nitrogen atoms and not more than 8 carbon atoms.

EP-A 0 068 192 describes a process for preparing abrasion-resistant coated catalysts. Binders recommended are glucose and urea.

DE-A 22 38 067 discloses a supported catalyst whose V₂O₅/TiO₂-containing active composition is applied to support bodies with the aid of a vinyl acetate-vinyl laurate copolymer dispersion containing 25% by weight of vinyl laurate. Copolymer dispersions containing vinyl laurate are specialty dispersions which are not available in large quantities and whose use increases the raw materials costs for the catalyst.

It is an object of the present invention to provide a catalyst for gas-phase oxidations, which is produced using commercial copolymer dispersions and has a high activity in respect of the gas-phase oxidation to be catalyzed.

We have now found that the monomer composition of the polymeric binder has a significant influence on the activity of the coated catalyst obtained.

The present invention provides a catalyst for gas-phase oxidations, which comprises an inert support and a catalytically active composition comprising transition metal oxides applied thereto, or a precatalyst for the catalyst, where the (pre)catalyst is obtainable by treatment of the inert support with an aqueous suspension solution of the transition metal oxides or their precursor compounds, which further comprises a binder dispersion, where the binder is a copolymer of an α-olefin and a vinyl C₂-C₄-carboxylate whose vinyl C₂-C₄-carboxylate content is at least 62 mol %, preferably from 63 to 95 mol %.

The reasons for the influence of the binder on the activity of the catalyst obtained are not completely clear. Catalysts for gas-phase oxidations comprise redox-active transition metal oxides such as V₂O₅. Possible catalytic centers here are vanadyl groups (V═O) or V—O—V— or V—O-support bridges. According to Grzybowska [B. Grzybowska-Swierkosz, “Vanadia-Titania Catalysts for Oxidation of o-Xylene and other Hydrocarbons”, Appl. Catal. A: General 157 (1997) 263-310], vanadyl groups are capable of abstracting hydrogen, but the oxygen is very strongly bound, so that insertion of the oxygen atom into a carbon-hydrogen bond of the substrate to be oxidized is not possible. On the other hand, V—O—V or V—O support groups are able to insert oxygen. According to Went et al. [G. T. Went, L.-J. Leu, A. T. Bell, “Quantitative Structural Analysis of Dispersed Vanadia Species in TiO₂ (Anatase)-Supported V₂O₅” J. Catal. 134 (1992) 479-491], terminal oxygen atoms are more readily reduced by H₂. As shown in the examples below, the catalysts of the present invention have a diminished H₂ uptake in temperature-programmed reduction (TPR); the proportion of monomeric vanadyl species (having V═O groups) has obviously been decreased to produce more polymeric vanadyl species. Presumably, complexation of the transition metal species by the binder used according to the present invention occurs, which modifies the way in which deposition on the support occurs.

According to the present invention, a copolymer of an α-olefin and a vinyl C₂-C₄-carboxylate having a high vinyl C₂-C₄-carboxylate content is used as binder (the “content” of a particular monomer in a copolymer refers to the content of copolymerized units of the monomer). The copolymers are generally random copolymers. Suitable vinyl C₂-C₄-carboxylates are, in particular, vinyl acetate and vinyl propionate, of which vinyl acetate is particularly preferred. Possible comonomers are α-olefins having from 2 to 20 carbon atoms, in particular ethylene, propylene, butene, hexene or octene, of which ethylene is preferred. Ethylene-vinyl acetate copolymers are particularly preferred, especially those consisting of from 63 to 70 mol % of vinyl acetate and from 37 to 30 mol % of ethylene.

The binders used according to the present invention are commercially available as aqueous dispersions having a solids content of, for example, from 35 to 65% by weight. The amount of such binder dispersions used is generally from 2 to 45% by weight, preferably from 5 to 35% by weight, particularly preferably from 7 to 20% by weight, based on the weight of the solution or suspension of the transition metal oxides or their precursor compounds. The content of dissolved and/or suspended transition metal oxides or their precursor compounds in the solution or suspension is generally from 20 to 50% by weight.

In the calcined state, the catalytically active composition preferably comprises, based on the total amount of catalytically active composition, from 1 to 40% by weight of vanadium oxide, calculated as V₂O₅, and from 60 to 99% by weight of titanium dioxide, calculated as TiO₂. The catalytically active composition can further comprise up to 1% by weight of a cesium compound, calculated as Cs, up to 1% by weight of a phosphorus compound, calculated as P, and up to 10% by weight of antimony oxide, calculated as Sb₂O₃.

Apart from the optional additives cesium and phosphorus, it is in principle possible for small amounts of many other oxidic compounds which act as promoters to influence the activity and selectivity of the catalyst, for example by decreasing or increasing its activity, to be present in the catalytically active composition. Promoters of this type are, for example, alkali metal oxides, in particular the abovementioned cesium oxide and also lithium oxide, potassium oxide and rubidium oxide, thallium(I) oxide, aluminum oxide, zirconium oxide, iron oxide, nickel oxide, cobalt oxide, manganese oxide, tin oxide, silver oxide, copper oxide, chromium oxide, molybdenum oxide, tungsten oxide, iridium oxide, tantalum oxide, niobium oxide, arsenic oxide, antimony oxide and cerium oxide. Among this group, cesium is generally used as promoter.

Furthermore, among the promoters mentioned, preference is given, as additives, to the oxides of niobium and tungsten in amounts of from 0.01 to 0.50% by weight, based on the catalytically active composition. As additives which increase the activity but reduce the selectivity, it is possible to use, in particular, oxidic phosphorus compounds, especially phosphorus pentoxide.

The catalytically active composition can also be applied in two or more layers, with, for example, the inner layer or layers having an antimony oxide content of up to 15% by weight and the outer layer having an antimony oxide content which has been reduced by from 50 to 100%. In general, the inner layer of the catalyst is phosphorus-containing and the outer layer is low in phosphorus or phosphorus-free.

The layer thickness of the catalytically active composition is generally from 0.02 to 0.2 mm, preferably from 0.05 to 0.15 mm. The proportion of active composition in the catalyst is usually from 5 to 25% by weight, mostly from 7 to 15% by weight. The titanium dioxide used advantageously consists of a mixture of a TiO₂ having a BET surface area of from 5 to 15 m²/g and a TiO₂ having a BET surface area of from 15 to 50 m²/g. It is also possible to use a titanium dioxide having a BET surface area of from 5 to 50 m²/g, preferably from 13 to 28 m²/g. As inert support material, it is possible to use virtually all support materials known from the prior art which are advantageously used in the production of coated catalysts for the oxidation of aromatic hydrocarbons to aldehydes, carboxylic acids and/or carboxylic anhydrides, for example quartz (SiO₂), porcelain, magnesium oxide, tin dioxide, silicon carbide, rutile, alumina (Al₂O₃), aluminum silicate, steatite (magnesium silicate), zirconium silicate, cerium silicate or mixtures of these support materials. The support material is generally nonporous. The expression “nonporous” in this context means “nonporous except for technically ineffective amounts of pores”, since a small number of pores may be technically unavoidable in a support material which should ideally contain no pores. As advantageous support materials, particular emphasis should be given to steatite and silicon carbide. The shape of the support material is generally not critical for the precatalysts and coated catalysts of the present invention. For example, catalyst supports in the form of spheres, rings, pellets, spirals, tubes, extrudates or granules can be used. The dimensions of these catalyst supports correspond to those of the catalyst supports customarily used for producing coated catalysts for the gas-phase partial oxidation of aromatic hydrocarbons. Preference is given to steatite in the form of spheres having a diameter of from 3 to 6 mm or rings having an external diameter of from 5 to 9 mm and a length of from 3 to 8 mm and a wall thickness of from 1 to 2 mm.

The application of the individual layers of the coated catalyst can be carried out using any methods known per se, e.g. by spraying solutions or suspensions onto the support in a coating drum or coating with a solution or suspension in a fluidized bed.

Coating of the catalyst support with the catalytically active composition is generally carried out at coating temperatures of from 20 to 500° C., with coating being able to be carried out in the coating apparatus under atmospheric pressure or under reduced pressure. Coating is generally carried out at from 0° C. to 200° C., preferably from 20 to 150° C., in particular at from room temperature to 120° C.

As a result of the precatalyst obtained in this way being treated thermally at from ≧200 to 500° C., the binder is removed from the applied layer by thermal decomposition and/or combustion. The thermal treatment is preferably carried out in situ in the gas-phase oxidation reactor.

In preferred embodiments, the catalysts of the present invention display, after thermal decomposition and/or combustion of the binder has been carried out (e.g. after calcination at 400° C. for four hours), an H₂ consumption of less than 5.5 mol/mol of vanadium (mol of H₂ per mol of vanadium present in the catalyst), preferably less than 5.0 mol/mol of vanadium, when they are heated from 25 to 923 K in a hydrogen-containing stream of inert gas.

The catalysts of the present invention are generally suitable for the gas-phase oxidation of aromatic C₆-C₁₀-hydrocarbons such as benzene, the xylenes, toluene, naphthalene or durene (1,2,4,5-tetramethylbenzene) to carboxylic acids and/or carboxylic anhydrides, e.g. maleic anhydride, phthalic anhydride, benzoic acid and/or pyromellitic dianhydride.

In particular, the novel coated catalysts make it possible to achieve a significant increase in the selectivity and yield in the preparation of phthalic anhydride.

For this purpose, the catalysts produced according to the present invention are installed in reaction tubes which are thermostatted from the outside to the reaction temperature, for example by means of salt melts, and the reaction gas is passed over the catalyst bed prepared in this way at generally from 300 to 450° C., preferably from 320 to 420° C. and particularly preferably from 340 to 400° C., and a gauge pressure of generally from 0.1 to 2.5 bar, preferably from 0.3 to 1.5 bar, at a space velocity of generally from 750 to 5000 h⁻¹.

The reaction gas supplied to the catalyst is generally produced by mixing a gas which comprises molecular oxygen and may, in addition to oxygen, further comprise suitable reaction moderators and/or diluents such as steam, carbon dioxide and/or nitrogen with the aromatic hydrocarbon to be oxidized, with the gas comprising molecular oxygen generally being able to comprise from 1 to 100 mol %, preferably from 2 to 50 mol % and particularly preferably from 10 to 30 mol %, of oxygen, from 0 to 30 mol %, preferably from 0 to 10 mol %, of water vapor and from 0 to 50 mol %, preferably from 0 to 1 mol %, of carbon dioxide, balance nitrogen. To produce the reaction gas, the gas comprising molecular oxygen is generally loaded with from 30 g to 150 g of the aromatic hydrocarbon to be oxidized per standard m³ of gas.

The gas-phase oxidation is advantageously carried out in two or more zones, preferably two zones, of the catalyst bed present in the reaction tube which are thermostatted to different reaction temperatures, for example using reactors having separate salt baths. If the reaction is carried out in two reaction zones, the reaction zone nearest the inlet for the reaction gas, which generally makes up from 30 to 80 mol % of the total catalyst volume, is generally thermostatted to a reaction temperature which is from 1 to 20° C. higher, preferably from 1 to 10° C. higher and in particular from 2 to 8° C. higher, than the reaction zone nearest the gas outlet. As an alternative, the gas-phase oxidation can also be carried out at one reaction temperature without division into temperature zones.

Regardless of the temperature structuring, it has been found to be particularly advantageous for catalysts which differ in their catalytic activity and/or chemical nature of their active composition to be used in the abovementioned reaction zones of the catalyst bed. When two reaction zones are employed, the catalyst used in the first reaction zone, i.e. the reaction zone nearest the inlet for the reaction gas, has a catalytic activity which is somewhat lower than that of the catalyst present in the second reaction zone, i.e. the reaction zone nearest the gas outlet. In general, the reaction is controlled via the temperature setting so that the major part of the aromatic hydrocarbon present in the reaction gas is reacted at maximum yield in the first zone.

Preference is given to using three- to five-zone catalyst systems, in particualr three- and four-zone catalyst systems.

In a preferred embodiment of a three-zone catalyst system, the catalysts have the following compositions:
for the first, uppermost zone (zone a)):
from 7 to 10% by weight of active composition, based on the total catalyst, where this active composition comprises:
from 6 to 11% by weight of vanadium (calculated as V₂O₅)
from 0 to 3% by weight of antimony trioxide,
from 0.1 to 1% by weight of an alkali (calculated as alkali metal), in particular cesium oxide,
and titanium dioxide in the anatase modification having a BET surface area of from 5 to 15 m²/g as balance to 100% by weight,
for the second, middle zone (zone b)):
from 7 to 12% by weight of active composition, based on the total catalyst, where this active composition comprises:
from 5 to 13% by weight of vanadium (calculated as V₂O₅)
from 0 to 3% by weight of antimony trioxide,
from 0 to 0.4% by weight of an alkali (calculated as alkali metal), in particular cesium oxide,
from 0 to 0.4% by weight of phosphorus pentoxide (calculated as P)
and titanium dioxide in the anatase modification, if desired as in zone a), as balance to 100% by weight,
for the third, bottommost zone (zone c)):
from 8 to 12% by weight of active composition, based on the total catalyst, where this active composition comprises:
from 5 to 30% by weight of vanadium (calculated as V₂O₅)
from 0 to 3% by weight of antimony trioxide
from 0 to 0.3% by weight of an alkali (calculated as alkali metal), in particular cesium oxide
from 0.05 to 0.4% by weight of phosphorus pentoxide (calculated as P)
and titanium dioxide, in particular in the anatase modification, if desired as in zone a), as balance to 100% by weight,

In a preferred embodiment of a four-zone catalyst system, the catalysts have the following compositions:
for the first zone (zone a)):
from 7 to 10% by weight of active composition, based on the total catalyst, where this active composition comprises:
from 6 to 11% by weight of vanadium (calculated as V₂O₅),
from 0 to 3% by weight of antimony trioxide,
from 0.1 to 1% by weight of an alkali (calculated as alkali metal), in particular cesium oxide,
and titanium dioxide in the anatase modification having a BET surface area of from 5 to 15 m²/g as balance to 100% by weight,
for the second zone (zone b1)):
from 7 to 12% by weight of active composition, based on the total catalyst, where this active composition comprises:
from 4 to 15% by weight of vanadium (calculated as V₂O₅),
from 0 to 3% by weight of antimony trioxide,
from 0.1 to 1% by weight of an alkali (calculated as alkali metal), in particular cesium oxide,
from 0 to 0.4% by weight of phosphorus pentoxide (calculated as P)
and titanium dioxide in the anatase modification, if desired as in zone a), as balance to 100% by weight,
for the third zone (zone b2)):
from 7 to 12% by weight of active composition, based on the total catalyst, where this active composition comprises:
from 5 to 15% by weight of vanadium (calculated as V₂O₅),
from 0 to 3% by weight of antimony trioxide,
from 0 to 0.4% by weight of an alkali (calculated as alkali metal), in particular cesium oxide,
from 0 to 0.4% by weight of phosphorus pentoxide (calculated as P)
and titanium dioxide in the anatase modification, if desired as in zone a), as balance to 100% by weight,
for the fourth zone (zone c)):
from 8 to 12% by weight of active composition, based on the total catalyst, where this active composition comprises:
from 5 to 30% by weight of vanadium (calculated as V₂O₅),
from 0 to 3% by weight of antimony trioxide,
from 0.05 to 0.4% by weight of phosphorus pentoxide (calculated as P)
and titanium dioxide in the anatase modification, if desired as in zone a), as balance to 100% by weight.

In general, the catalyst zones a), b), c) and/or d) can also be arranged so that they consist of two or more subzones. These intermediate zones advantageously have intermediate catalyst compositions.

Instead of delineated zones of the various catalysts, it is also possible to achieve a pseudocontinuous transition of the zones and an effectively uniform increase in the activity by using a mixture of the successive catalysts at the transition from one zone to the next zone.

The bed length of the first catalyst zone preferably makes up more than 30-80% of the total catalyst fill height in the reactor. The bed height of the first two or first three catalyst zones advantageously makes up more than 60-95% of the total catalyst fill height. Typical reactors have a fill height of from 250 cm to 350 cm. The catalyst zones can also, if desired, be distributed over a plurality of reactors.

If desired, a downstream finishing reactor as described, for example, in DE-A 198 07 018 or DE-A 20 05 969 can be additionally provided for the preparation of phthalic anhydride. The catalyst used is preferably a catalyst which is even more active than the catalyst of the last zone.

If the preparation of PA is carried out using the catalysts of the present invention and a plurality of reaction zones in which different catalysts are present, the novel coated catalysts can be used in all reaction zones. However, considerable advantages over conventional processes can generally be achieved even when coated catalysts according to the present invention are used in only one of the reaction zones of the catalyst bed, for example the first reaction zone, or in the first two reaction zones and coated catalysts produced in a conventional way are employed in the remaining reaction zones. The hot spot temperatures prevailing in the first reaction zone(s) are higher than in the downstream reaction zones; in this/these zone(s), the major part of the starting hydrocarbon is oxidized to the desired oxidation product and/or intermediates, so that the advantages of the catalysts of the present invention are particularly evident in the first stage or the first and second stages.

The invention is illustrated by the following examples.

A. Production of Catalysts

Upper Zone Catalysts R1.1 to R1.4

34.64 g of titanium dioxide (BET surface area 9 m²/g), 64.33 g of titanium dioxide (BET surface area 20 m²/g), 7.82 g of vanadium pentoxide, 2.60 g of antimony oxide and 0.444 g of cesium carbonate were suspended in 650 ml of deionized water and the mixture was stirred for 18 hours. The binders indicated in Table 1 below were added to this suspension. The suspension obtained was subsequently sprayed onto 1200 g of steatite (magnesium silicate) in the form of rings (7×7×4 mm, external diameter, length, internal diameter) and dried. The weight of the applied coating was 8.0% of the total weight of the finished catalyst. The catalytically active composition applied in this way comprised, after calcination at 400° C. for four hours, 7.12% by weight of vanadium (calculated as V₂O₅), 2.37% by weight of antimony (calculated as Sb₂O₃), 0.33% by weight of cesium (calculated as Cs) and 90.1% by weight of titanium dioxide.

TABLE 1

| | Binder | Amount of |
|---|---|---|
| Cat. | (molar comonomer ratio) | binder [g] |
| R1.1 | Vinyl acetate-ethylene copolymer | 25 |
| | (63:37) | |
| R1.2 | Vinyl acetate-ethylene-copolymer | 25 |
| | (67:33) | |
| R1.3* | Vinyl acetate-ethylene-copolymer | 25 |
| | (60:40) | |
| R1.4* | Hydroxyethylcellulose | 4 |

*Comparative Examples

Middle Zone Catalyst R2:

34.32 g of titanium dioxide (BET surface area: 9 m²/g), 102.90 g of titanium dioxide (BET surface area 20 m²/g), 11.0 g of vanadium pentoxide. 2.30 g of ammonium dihydrogenphosphate, 3.66 g of antimony oxide and 0.19 g of cesium carbonate were suspended in 650 ml of water and the mixture was stirred for 18 hours. 50 g of organic binder consisting of a copolymer of vinyl acetate and vinyl laurate in the form of a 50% strength by weight dispersion were added to this suspension. The suspension obtained was subsequently sprayed onto 1200 g of steatite (magnesium silicate) in the form of rings (7×7×4 mm, external diameter, length, internal diameter) and dried. The weight of the applied coating was 10.0% of the total weight of the finished catalyst. The catalytically active composition applied in this way comprised, after calcination at 400° C. for four hours, 7.12% by weight of vanadium (calculated as V₂O₅), 0.40% by weight of phosphorus (calculated as P), 2.37% by weight of antimony (calculated as Sb₂O₃), 0.10% by weight of cesium (calculated as Cs) and 88.91% by weight of titanium dioxide.

Lower Zone Catalyst R3:

24.56 g of titanium dioxide (BET surface area: 9 m²/g), 73.67 g of titanium dioxide (BET surface area: 30 m²/g), 24.99 g of vanadium pentoxide and 1.71 g of ammonium dihydrogenphosphate were suspended in 650 ml of water and the mixture was stirred for 18 hours. 58.6 g of organic binder consisting of a copolymer of vinyl acetate and ethylene (molar ratio=63:37) in the form of a 50% strength by weight dispersion were added to this suspension. The suspension obtained was subsequently sprayed onto 1200 g of steatite (magnesium silicate) in the form of rings (7×7×4 mm, external diameter, length, internal diameter) and dried. The weight of the applied coating was 9.3% of the total weight of the finished catalyst. The catalytically active composition applied in this way comprised, after calcination at 400° C. for four hours, 19.81% by weight of vanadium (calculated as V₂O₅), 0.45% by weight of phosphorus (calculated as P) and 78.63% by weight of titanium dioxide.

B. Catalyst Test of the Catalysts R1.1 to R1.4 by Means of Temperature-Programmed Reduction (TPR)

The temperature-programmed reduction was carried out by heating the sample in a stream of hydrogen/inert gas at a constant temperature increase per unit time. An apparatus whose construction was based on the proposals of Monti und Baiker [D.A.M. Monti, A. Baiker, “Temperature-Programmed Reduction. Parametric Sensitivity and Estimation of Kinetic Parameters”, J. Catal. 83 (1983) 323- 335] was used. The samples were installed as a loose bed between two glass wall plugs in a U-shaped glass tube. The U-tube was located in a ceramic tube furnace. The catalyst was firstly calcined at 400° C. for 4 hours while passing air through the tube (excess of oxygen).

After cooling, the sample was heated at a heating ramp of 10 K/min from ambient temperature to a final temperature of 923 K. The sample temperature was measured in a thermocouple sheath close to the bed and was recorded at intervals of 2 s. A stream of hydrogen/argon containing 4.2% of hydrogen was passed through the U-tube. The hydrogen content in the offgas was determined by means of a thermal conductivity detector. The thermal conductivity detector was calibrated by means of the reduction of CuO to Cu(0). The hydrogen consumption was recorded as a function of temperature. The total H₂ consumption over the temperature interval examined was determined by integration.

TABLE 2

| | | H₂ consumption |
|---|---|---|
| | H₂ consumption | [mol/mol |
| Cat. | [mmol/g] | of V] |
| R1.1 | 0.89 | 4.6 |
| R1.2 | 0.89 | 4.6 |
| R1.3* | 1.31 | 6.7 |
| R1.4* | 1.17 | 6 |

*Comparative Examples

C. Preparation of Phthalic Anhydride (Structured Bed with Two Catalyst Zones of R1 and R2)

From the bottom upward, 1.30 m of the catalyst R2 and 1.50 m of the catalyst R1.1 or R1.4 were introduced into an iron tube having a length of 3.3 m and an internal diameter of 25 mm. To regulate the temperature, the iron tube was surrounded by a salt melt, and a 2 mm thermocouple sheath containing a movable thermocouple was employed for measuring the catalyst temperature. 4.0 standard m³/h of air laden with about 40 g of 99.3% by weight pure o-xylene per standard m³ were passed through the tube from the top downwards. The results shown in Table 3 below were obtained. “PA yield” is parts by weight of phthalic anhydride obtained per 100 parts by weight of pure o-xylene.

TABLE 3

Cat.

| | R1.1/R2 | R1.4/R2 |
|---|---|---|
| Loading [g/standard m³] | 46 | 37 |
| Salt bath temperature [° C.] | 400 | 400 |
| Hot spot temperature in | 464 | 432 |
| upper zone [° C.] | | |
| Average PA yield | 106.6 | 104.3 |
| Residual o-xylene [% by | 0.01 | 0.01 |
| weight] | | |
| Phthalide [% by weight] | 0.11 | 0.80 |

It can be seen that a higher PA yield and better product quality are obtained when using the catalyst R1.1 according to the present invention.

D. Preparation of Phthalic Anhydride (Structured Bed with Three Catalyst Zones of R1, R2 and R3)

From the bottom upward, 0.70 m of the catalyst R3, 0.60 m of the catalyst R2 and 1.50 m of the catalyst R1.1 were introduced into an iron tube having a length of 3.85 m and an internal diameter of 25 mm. To regulate the temperature, the iron tube was surrounded by a salt melt, and a 2 mm thermocouple sheath containing a movable thermocouple was employed for measuring the catalyst temperature. 4.0 standard m³/h of air laden with about 70 g of 99.3% by weight pure o-xylene per standard m³ were passed through the tube from the top downwards. The results shown in Table 4 below were obtained.

TABLE 4

| | Cat. |
|---|---|
| | R1.1/R2/R3 |
| Loading [g/standard m³] | 70 |
| Salt bath temperature [° C.] | 365 |
| Hot spot temperature in | 432 |
| upper zone [° C.] | |
| Average PA yield | 112.5 |
| Residual o-xylene [% by | <0.01 |
| weight] | |
| Phthalide [% by weight] | 0.01 |

1. A catalyst for gas-phase oxidations prepared by a process comprising contacting a support with an aqueous suspension or solution comprising a transition metal oxides composition or their precursor compounds, wherein the suspension or solution contains a binder dispersion consisting essentially of a copolymer of an α-olefin and a vinyl-C₂-C₄-carboxylate whose vinyl C₂-C₄-carboxylate content is at least 62 mol %.

2. A catalyst as claimed in claim 1, wherein the vinyl C₂-C₄-carboxylate copolymer is a vinyl acetate copolymer.

3. A catalyst as claimed in claim 2, wherein the vinyl acetate copolymer is an ethylene-vinyl acetate copolymer.

4. A catalyst as claimed in claim 3, wherein the ethylene-vinyl acetate copolymer comprises from 63 to 70 mol % of vinyl acetate and from 37 to 30 mol % of ethylene.

5. A catalyst as claimed in claim 1, wherein the transition metal oxides composition comprises from 1 to 40% by weight of vanadium oxide, calculated as V₂O₅, and from 60 to 99% by weight of titanium dioxide, calculated as TiO₂.

6. A catalyst as claimed in claim 5, wherein the transition metal oxides composition further comprises up to 1% by weight of a cesium compound, calculated as Cs, up to 1% by weight of a phosphorus compound, calculated as P, or up to 10% by weight of antimony oxide, calculated as Sb₂O₃.

7. A process for preparing aldehydes, carboxylic acids and/or carboxylic anhydrides, comprising providing a gaseous stream comprising an aromatic hydrocarbon and a gas comprising molecular oxygen, and contacting the gaseous stream with a catalyst as claimed in claim 1 at an at elevated temperature.

8. A process as claimed in claim 7, wherein the catalyst is produced in situ from a precatalyst at an elevated temperature sufficient to decompose the copolymer.

9. A process as claimed in claim 7, wherein the aromatic hydrocarbon is selected from o-xylene, naphthalene or a mixture of o-xylene and naphthalene.

10. A precatalyst comprising transition metal oxides attached to a support with a binder, wherein the binder consists essentially of a copolymer of an α-olefin and a vinyl-C₂-C₄-carboxylate, wherein the vinyl C₂-C₄-carboxylate content is at least 62 mol %.

11. The precatalyst according to claim 10, wherein the copolymer is an ethylene-vinyl acetate copolymer comprising from 63 to 70 mol % of vinyl acetate and from 37 to 30 mol % of ethylene.

12. The precatalyst according to claim 10, wherein the transition metal oxides comprises from 1 to 40% by weight of vanadium oxide, calculated as V₂O₅, and from 60 to 99% by weight of titanium dioxide, calculated as TiO₂.

13. The precatalyst according to claim 12, wherein the transition metal oxides are disposed in at least a two zone catalyst system, wherein the upstream zone of the catalyst system contains an upstream pre-catalyst that contains less vanadium oxide relative to the amount of titanium oxide than a downstream pre-catalyst.

14. The precatalyst according to claim 13, wherein the upstream precatalyst further comprises up to 10% by weight of antimony oxide, calculated as Sb₂O₃, and the down stream catalyst comprises up to 1% by weight of a phosphorus compound, calculated as P.

15. A binder composition in combination with transition metal oxides, the binder composition consisting essentially of a copolymer of an α-olefin and a vinyl-C₂-C₄-carboxylate, wherein the vinyl C₂-C₄-carboxylate content is at least 62 mol %.

16. The binder composition according to claim 15, wherein the copolymer is an ethylene-vinyl acetate copolymer comprises from 63 to 70 mol % of vinyl acetate and from 37 to 30 mol % of ethylene.

17. The binder composition according to claim 15, wherein the transition metal oxides comprises from 1 to 40% by weight of vanadium oxide, calculated as V₂O₅, and from 60 to 99% by weight of titanium dioxide, calculated as TiO₂.
